(19) United States
(12) Patent Application Publication
(10) Pub. No.: US 2024/0238392 A1
(43) Pub. Date: Jul. 18, 2024

(54) TREATMENT OF ACUTE HEART FAILURE USING NITROUS OXIDE AND CHRONIC HEART FAILURE USING BOTULINUM TOXIN

(71) Applicant: Penland Foundation, Beaumont, TX (US)

(72) Inventor: Roland M. Williams, Beaumont, TX (US)

(21) Appl. No.: 18/392,662

(22) Filed: Dec. 21, 2023

Related U.S. Application Data
(60) Provisional application No. 63/479,703, filed on Jan. 12, 2023.

(51) Int. Cl. A61K 38/48; A61K 9/00; A61K 31/519; A61K 31/714; A61K 33/00; A61P 9/04

(52) U.S. Cl. CPC A61K 38/4893; A61K 9/0019; A61K 9/007; A61K 9/0085; A61K 31/519; A61K 31/714; A61K 33/00; A61P 9/04; C12Y 304/24069

(57) ABSTRACT

Provided is a method of treating chronic heart failure in a patient in need thereof. The method administering a botulinum toxin to the patient, thereby treating chronic heart failure. Also provided is a method of treating acute heart failure in a patient in need thereof. The method includes administering nitrous oxide and oxygen and B-12 and/or folic acid to the patient, thereby treating acute heart failure.

### CROSS-REFERENCE TO RELATED APPLICATION

This application claims the benefit of U.S. Provisional Application No. 63/479,703, filed Jan. 12, 2023, which is incorporated herein in its entirety.

### BACKGROUND

### Technical Field

The present disclosure relates to methods for treating, alleviating, and/or preventing the acute, and chronic activation of the cytokine system, which is a major factor in the pathology of acute and chronic heart failure. Conditions involved, but not limited to, are heart attacks, chronic heart failure, cardiomyopathy virus infection and other conditions causing acute or chronic elevation of cytokines in the heart. The present disclosure also generally relates to a mechanism of the development of acute and chronic heart failure—the pathological acute and chronic activation of the cytokine system, which results in acute or chronic heart failure.

### Background

Cytokine production is a normal part of infection control and damage repair. However, if overproduced, acutely or chronically, they can damage or destroy normal heart tissue. Substance P activates the NK-1-3 receptor on immune cells triggering the release of cytokines. Nitrous oxide could mitigate the overproduction of acute levels of substance P. This can suppress and control the acute cytokine damage to normal heart tissue in conditions such as but not limited to, heart attack, viral infection, or injuries to the heart. Botulinum toxin by its ability to selectively stop only the chronic overproduction of substance P in the neurostructural cells of the sensory ganglia can mitigate chronic inflammation and the resulting loss of heart tissue and function in chronic heart failure (CHF).

### SUMMARY

The present disclosure in some embodiments is related to a method of treating chronic heart failure in a patient in need thereof. The method may include administering a botulinum toxin to the patient by subcutaneous or intradermal injection, 1-4 units to and/or around the vicinity of a trigeminal nerve, 1-4 units to and/or around the vicinity of a cervical nerve, lateral to the patient's spine, 1-4 units to and/or around the vicinity of a thoracic nerve, lateral to the spine, 1-4 units to and/or around the vicinity of a lumbar nerve, lateral to the spine, and/or 1-4 units to and/or around the vicinity of a sacral nerve, lateral to the spine, thereby treating chronic heart failure.

In some embodiments, the chronic heart failure may be caused by coronary artery disease, a previous myocardial infarction (heart attack), high blood pressure, atrial fibrillation, valvular heart disease, excess alcohol use, infection, smoking, and idiopathic cardiomyopathy.

In some embodiments, the trigeminal nerve may be selected from the group consisting of an ophthalmic nerve, maxillary nerve, mandibular nerve, supra orbital nerve, supra trochlear nerve, infraorbital nerve, lacrimal nerve, nasociliary nerve, superior alveolar nerve, buccal nerve, lingual nerve, inferior alveolar nerve, mental nerve, an auriculotemporal nerve, lesser occipital nerve, a greater occipital nerve and a combination thereof.

In some embodiments, the cervical nerve may be selected from the group consisting of a c-2 nerve, c-3 nerve, c-4 nerve, c-5 nerve, c-6 nerve, c-7 nerve, c-8 nerve and a combination thereof.

In some embodiments, the thoracic nerve may be selected from the group consisting of a t-2 nerve, t-3 nerve, t-5 nerve, t-6 nerve, t-7 nerve, t-8 nerve, t-9 nerve, t-10 nerve, t-11 nerve, t-12 nerve and a combination thereof.

In some embodiments, the lumbar nerve may be selected from the group consisting of a 1-1 nerve, 1-2 nerve, 1-3 nerve, 1-4 nerve, 1-5 nerve and a combination thereof.

In some embodiments, the sacral nerve may be selected from the group consisting of a s-1 nerve, s-2 nerve, s-3 nerve, s-4 nerve, s-5 nerve and a combination thereof.

In some embodiments, the botulinum toxin may be selected from the group consisting of botulinum toxin type A, botulinum toxin type B, botulinum toxin type C, botulinum toxin type D, botulinum toxin type E, botulinum toxin type F and botulinum toxin type G, a fragment thereof, a hybrid thereof, a chimera thereof, and a combination thereof.

In some embodiments, a therapeutically effective amount of the botulinum toxin may be 1-60 units.

For example, a total dosage of the botulinum toxin to an adult who weighs about 150 lbs. is less than or equal to about 50 units, and the total dosage of the botulinum toxin in an adult is adjusted for weight.

In some embodiments, each of the subcutaneous or intradermal injections may be bilateral.

The present disclosure in some embodiments is also related to a method of treating acute heart failure in a patient in need thereof. The method may include administering nitrous oxide and oxygen to the patient by inhalation, thereby treating acute heart failure.

In some embodiments, a composition of the nitrous oxide and oxygen may comprise from about 1% nitrous oxide/about 99% oxygen to about 70% nitrous oxide/about 30% oxygen. Alternatively, the composition of the nitrous oxide and oxygen may comprise from about 40% nitrous oxide/about 60% oxygen to about 50% nitrous oxide/about 50% oxygen. Alternatively, the composition of the nitrous oxide and oxygen may comprise about 50% nitrous oxide/about 50% oxygen.

In some embodiments, the nitrous oxide and oxygen may be provided to an adult who weighs about 150 lbs. for about between 1 minute and about 1 hour, every about 4-6 hours. Preferably, the nitrous oxide and oxygen may be provided to an adult who weighs about 150 lbs. for about 20 minutes, every about 4-6 hours. The nitrous oxide and oxygen may be provided by continuous administration over the period of time.

A composition, duration, an interval, and a total amount of the inhaled nitrous oxide and oxygen provided to an adult, or a child may be adjusted for age, weight, or a combination thereof.

The method may further comprise administering B-12 and/or folic acid to the patient.

### DETAILED DESCRIPTION OF THE PREFERRED EMBODIMENTS

Further in relation to this, before explaining at least the preferred embodiments of the disclosure in greater detail, it is to be understood that the disclosure is not limited in its application to the details of construction and to the arrangements of the components set forth in the following description. It would be understood by those of ordinary skill in the art that embodiments beyond those described herein are contemplated, and the embodiments can be practiced and carried out in a plurality of different ways. Also, it is to be understood that the terminology used herein is for the purpose of description and should not be regarded as a limiting factor.

Unless otherwise defined, the terms used herein refer to that which the ordinary artisan would understand such term to mean based on the contextual use of such term herein. To the extent that the meaning of a term used herein as understood by the ordinary artisan based on the contextual use of such term differs in any way from any particular dictionary definition of such ern, it is intended that the meaning of the term as understood by the ordinary artisan will prevail.

As used herein, the term “about” means approximately or nearly and in the context of a numerical value or range set forth herein means 10%, of the numerical value or range recited or claimed.

The term “treating” includes delaying, alleviating, mitigating or reducing the intensity, progression, or worsening of one or more attendant symptoms of a disorder or condition and/or alleviating, mitigating or impeding one or more causes of a disorder or condition. Treatment under the present disclosure may be a preventative treatment, prophylactic treatment, remission of treating or ameliorating treatment.

The term “therapeutically effective amount” or “therapeutically effective dose” refers to the amount of a composition, compound, therapy, or course of treatment that, when administered to an individual for treating a disorder or disease, is sufficient to effect such treatment for the disorder or disease. The “therapeutically effective amount” will vary depending on the composition, the compound, the therapy, the course of treatment, the disorder or disease and its severity and the age, weight, etc., of the individual to be treated.

The term “unit” refers to the amount of botulinum toxin needed to kill 50% of a group of 18-20 gm female Swiss-Webster mice given the injection intraperitoneally.

The term “vicinity of a nerve” refers to anywhere on the dermatome involved with the nerve.

As used herein, “consists essentially of” when used in conjunction with a composition means excluding other materials that contribute to mitigating cytokine overproduction, thereby treating heart failure that have resulted from the overproduction of cytokines. The objective of administering botulinum toxin/inhaled anesthetics is to treat the conditions by mitigating cytokine overproduction. With the language, other materials that contribute to the treatment that materially affect the basic and novel characteristics of the disclosure are not required and are potentially counterproductive because they may offset the treatment effect of botulinum toxin/inhaled anesthetics. In other words, the meaning of “consists essentially of” is tied to the objective and excludes materials (that contribute to the treatment) that are pharmaceutically active for the treatment and materially mitigate cytokine overproduction and thereby affecting the treatment of the conditions. Small traces that have little or no effect to the treatment as part of the embodiments of the presentation disclosure may exist in a composition that consists essentially of botulinum toxin and/or inhaled anesthetic under the definition because it would not materially affect its function and/or objective.

In accordance with the principles of the present disclosure, use of botulinum toxin/inhaled anesthetics to treat heart failure is provided.

### Chronic Heart Failure

Chronic Heart Failure (CHF), previously known as congestive heart failure, and heart failure, occurs when the heart cannot properly pump sufficient amounts of blood to maintain blood flow, thereby meeting the body tissues' needs for metabolism.

### Statistics

Chronic Heart Failure is a common, costly, and potentially fatal condition, and is the leading cause of both hospitalization and readmission amongst older adults.

In 2015, it affected about 40 million people globally. Overall, around 2% of adults have heart failure, and in those over the age of 65, this increases to 6-10%. Rates are predicted to increase in the future. The risk of death is about 35% the first year after diagnosis, while by the second year, the risk of death is less than 10% for those who remain alive. This degree of risk of death comparable to some cancers. In the United Kingdom, the disease accounts for 5% of emergency hospital admissions.

### Types of Heart Failure

The two types of left ventricular heart failure—heart failure with reduced ejection fraction (HFrEF), and heart failure with preserved ejection fraction (HFpEF)—are based on whether the ability of the left ventricle to contract or to relax, is affected.

### Systolic Dysfunction (HFrEF)

The most common causes of systolic dysfunction (HFrEF, or Heart Failure with Reduced Ejection Fraction) are idiopathic dilated cardiomyopathy (DCM), coronary heart disease (ischemic), hypertension, and valvular disease. However, in up to 50% of cases, the cause is at least initially unknown (idiopathic dilated cardiomyopathy—DCM).

HFrEF occurs when the left ventricular ejection fraction (LVEF) is 40% or less and is accompanied by progressive left ventricular dilatation and adverse cardiac remodeling. Hypertension, obesity, coronary artery disease, diabetes mellitus, atrial fibrillation, and hyperlipidemia are highly prevalent in HFpEF (Heart Failure with Preserved Ejection Fraction) patients.

### Diastolic Dysfunction (HFpEF)

HFpEF occurs when the lower left chamber (left ventricle) is not able to fill properly with blood during the diastolic (filling) phase. The amount of blood pumped out to the body is less than normal.

Hypertension by far is the most important cause of HFpEF. In addition, conditions like hypertrophic obstructive cardiomyopathy, and restrictive cardiomyopathy are associated with significant diastolic dysfunction, leading to HFpEF. These cause heart failure by changing either the structure or the function of the heart.

### Causes, Severity and Symptoms

Common causes of chronic heart failure include coronary artery disease, including a previous myocardial infarction (heart attack), high blood pressure, atrial fibrillation, valvular heart disease, excess alcohol use, infection, smoking, and idiopathic cardiomyopathy.

The severity of the heart failure is graded by the severity of symptoms with exercise. Heart failure is the potential end stage of all diseases of the heart. In addition, viral infections of the heart can lead to inflammation of the muscular layer of the heart and subsequently contribute to the development of heart failure. Genetic predisposition plays an important role. If more than one cause is present, progression is more likely, and prognosis is worse. Heart damage can predispose a person to develop heart failure later in life and has many causes including systemic viral infections (e.g., HIV), chemotherapeutic agents such as daunorubicin, cyclophosphamide, trastuzumab and substance use disorders of substances such as alcohol, cocaine, and methamphetamine. An uncommon cause is exposure to certain toxins such as lead and cobalt. Additionally, infiltrative disorders such as amyloidosis and connective tissue diseases such as systemic lupus erythematosus have similar consequences. Obstructive sleep apnea (a condition of sleep wherein disordered breathing overlaps with obesity, hypertension, and/or diabetes) is regarded as an independent cause of heart failure. Recent reports from clinical trials have also linked variation in blood pressure to heart failure and cardiac changes that may give rise to heart failure.

Symptoms of heart failure commonly include shortness of breath, fatigue, and leg swelling. Shortness of breath usually gets exacerbated with exercise or while lying down and may wake the person at night. A limited ability to exercise can cause the condition to worsen. Chest pain, including angina, does not typically occur due to heart failure. Heart failure is a pathophysiological state in which cardiac output is insufficient to meet the needs of the body and lungs. The term “congestive heart failure” is often used, as one of the common symptoms is congestion, or build-up of fluid in a person's tissues and veins in the lungs or other parts of the body. Specifically, congestion takes the form of water retention and swelling (edema), both as peripheral edema (causing swollen limbs and feet) and as pulmonary edema (causing breathing difficulty), as well as ascites (swollen abdomen). Heart failure symptoms are traditionally divided into left- and right-sided, recognizing that the left and right ventricles of the heart supply different portions of the body's circulation, but people commonly have both sets of signs and symptoms.

Heart failure is caused by any condition that reduces the efficiency of the heart muscle, through damage or overloading. Over time, these increases in workload, which are mediated by long-term activation of neurohormonal systems such as the renin-angiotensin system, lead to fibrosis, dilation, and structural changes in the shape of the left ventricle from elliptical to spherical. The heart of a person with heart failure may have a reduced force of contraction due to overloading of the ventricle. In a normal heart, increased filling of the ventricle results in increased contraction force by the Frank-Starling law of the heart, and thus a rise in cardiac output. In heart failure, this mechanism fails, as the ventricle is loaded with blood to the point where heart muscle contraction becomes less efficient. This is due to reduced ability to cross-link actin and myosin filaments in over-stretched heart muscle.

### Pathophysiology

When it comes to the pathophysiology of heart failure, the adaptive mechanisms that may be adequate to maintain the overall contractile performance of the heart at relatively normal levels become maladaptive when trying to sustain adequate cardiac performance. The primary myocardial response is to chronically increased wall stress is myocyte hypertrophy, death due to apoptosis, and regeneration. This process eventually leads to remodeling, usually the eccentric type, and reduced cardiac output, causing a cascade of the neurohumoral and vascular mechanism. Decreased carotid baroreceptor stimulation and renal perfusion will activate the sympathetic nervous system and Renin-Angiotensin-Aldosterone system. Sympathetic nervous system activation will cause increased heart rate and inotropy, leading to myocardial toxicity. Renin-Angiotensin-Aldosterone system activation leads to vasoconstriction, increasing afterload (angiotensin II) and hemodynamic alterations, increasing preload (aldosterone). Both BNP and ANP are peptides released from the atria and ventricles in response to heart chamber pressure/volume expansion. These peptides promote natriuresis and vasodilatation. In addition, BNP inhibits the reabsorption of sodium in the proximal convoluted tubule. It also suppresses renin and aldosterone release. In patients with HFpEF there is impaired relaxation and increasing ventricle stiffness, leading to dysfunction in diastolic filling of the left ventricle. Patients with concentric left ventricular hypertrophy have a shift of the diastolic pressure volume curve to the left, leading to elevation in diastolic pressures, which leads to increased energy expenditure and oxygen demand and myocardial ischemia. These mechanisms will cause negative remodeling and worsen the left ventricular function, causing symptoms of heart failure.

### Diagnosis

Diagnosis is based on symptoms, physical findings, and echocardiography. Blood tests, electrocardiography, and chest radiography may be useful to determine the underlying cause. No diagnostic criteria have been agreed on as the gold standard for heart failure. The National Institute for Health and Care Excellence recommends measuring brain natriuretic peptide (BNP) followed by an ultrasound of the heart if positive. This is recommended in those with shortness of breath. In those with worsening heart failure, both a BNP and a troponin are recommended to help determine likely outcomes. One historical method of categorizing heart failure is by the side of the heart involved (left heart failure versus right heart failure). Right heart failure was thought to compromise blood flow to the lungs compared to left heart failure compromising blood flow to the aorta and consequently to the brain and the remainder of the body's systemic circulation. However, mixed presentations are common and left heart failure is a common cause of right heart failure. More accurate classification of heart failure type is made by measuring ejection fraction, or the proportion of blood pumped out of the heart during a single contraction. Ejection fraction is given as a percentage with the normal range being between 50 and 75%. The two types are: 1) Heart failure due to reduced ejection fraction (HFrEF): Synonyms no longer recommended are “heart failure due to left ventricular systolic dysfunction” and “systolic heart failure”. HFrEF is associated with an ejection fraction less than 40%. 2) Heart failure with preserved ejection fraction (HFpEF): Synonyms no longer recommended include “diastolic heart failure” and “heart failure with normal ejection fraction.” HFpEF occurs when the left ventricle contracts normally during systole, but the ventricle is stiff and does not relax normally during diastole, which impairs filling. Heart failure may also be classified as acute or chronic. Chronic heart failure is a long-term condition, usually kept stable by the treatment of symptoms. Acute decompensated heart failure is a worsening of chronic heart failure symptoms, which can result in acute respiratory distress. High-output heart failure can occur when there is increased cardiac demand that results in increased left ventricular diastolic pressure which can develop into pulmonary congestion (pulmonary edema). Several terms are closely related to heart failure and may be the cause of heart failure, but should not be confused with it. Cardiac arrest and asystole refer to situations in which no cardiac output occurs at all. Without urgent treatment, these result in sudden death. Myocardial infarction (“heart attack”) refers to heart muscle damage due to insufficient blood supply, usually as a result of a blocked coronary artery. Cardiomyopathy refers specifically to problems within the heart muscle, and these problems can result in heart failure. Ischemic cardiomyopathy implies that the cause of muscle damage is coronary artery disease. Dilated cardiomyopathy implies that the muscle damage has resulted in enlargement of the heart. Hypertrophic cardiomyopathy involves enlargement and thickening of the heart muscle. Echocardiography is commonly used to support a clinical diagnosis of heart failure. This modality uses ultrasound to determine the stroke volume (SV, the amount of blood in the heart that exits the ventricles with each beat), the end-diastolic volume (EDV, the total amount of blood at the end of diastole), and the SV in proportion to the EDV, a value known as the ejection fraction (EF). In pediatrics, the shortening fraction is the preferred measure of systolic function. Normally, the EF should be between 50 and 70%; in systolic heart failure, it drops below 40%. Echocardiography can also identify valvular heart disease and assess the state of the pericardium (the connective tissue sac surrounding the heart). Echocardiography may also aid in deciding what treatments will help the person, such as medication, insertion of an implantable cardioverter-defibrillator, or cardiac resynchronization therapy. Echocardiography can also help determine if acute myocardial ischemia is the precipitating cause and may manifest as regional wall motion abnormalities on echo. Chest X-rays are frequently used to aid in the diagnosis of CIF. In a person who is compensated, this may show cardiomegaly (visible enlargement of the heart), quantified as the cardiothoracic ratio (proportion of the heart size to the chest). In left ventricular failure, evidence may exist of vascular redistribution (upper lobe blood diversion or cephalization), Kerley lines, cuffing of the areas around the bronchi, and interstitial edema. Ultrasound of the lung may also be able to detect Kerley lines. An electrocardiogram (ECG/EKG) may be used to identify arrhythmias, ischemic heart disease, right and left ventricular hypertrophy, and presence of conduction delay or abnormalities (e.g. left bundle branch block). Although these findings are not specific to the diagnosis of heart failure, a normal ECG virtually excludes left ventricular systolic dysfunction. Blood tests routinely performed include electrolytes (sodium, potassium), measures of kidney function, liver function tests, thyroid function tests, a complete blood count, and often C-reactive protein if infection is suspected. An elevated brain natriuretic peptide (BNP) is a specific test indicative of heart failure. Additionally, BNP can be used to differentiate between causes of dyspnea due to heart failure from other causes of dyspnea. If myocardial infarction is suspected, various cardiac markers may be used. BNP is a better indicator than N-terminal pro-BNP for the diagnosis of symptomatic heart failure and left ventricular systolic dysfunction. In symptomatic people, BNP had a sensitivity of 85% and specificity of 84% in detecting heart failure; performance declined with increasing age. Hyponatremia, which is a low serum sodium concentration, is common in heart failure. Vasopressin levels are usually increased, along with renin, angiotensin II, and catecholamines to compensate for reduced circulating volume due to inadequate cardiac output. This leads to increased fluid and sodium retention in the body; the rate of fluid retention is higher than the rate of sodium retention in the body, this phenomenon causes hypervolemic hyponatremia. This phenomenon is more common in older women with low body mass. Severe hyponatremia can result in accumulation of fluid in the brain, causing cerebral edema and intracranial hemorrhage. Angiography is the X-ray imaging of blood vessels, which is done by injecting contrast agents into the bloodstream through a thin plastic tube (catheter) placed directly in the blood vessel. X-ray images are called angiograms. Heart failure may be the result of coronary artery disease, and its prognosis depends in part on the ability of the coronary arteries to supply blood to the myocardium (heart muscle). As a result, coronary catheterization may be used to identify possibilities for revascularization through percutaneous coronary intervention or bypass surgery. Other methods of diagnosis include algorithms, Framingham criteria, ESC algorithm, staging, and histopathology, which is done during autopsies.

### Treatment

Treatment depends on the severity and cause of the disease. In patients with chronic stable mild heart failure, treatment commonly consists of lifestyle modifications such as quitting smoking, exercise, dietary changes, and possibly medications. In those with heart failure due to left ventricular dysfunction, angiotensin converting enzyme inhibitors, angiotensin receptor blockers, or valsartan/sacubitril along with beta blockers are recommended. For those with severe disease, aldosterone antagonists, or hydralazine with a nitrate may be used. Diuretics are useful for preventing fluid retention and the resulting shortness of breath. Sometimes, depending on the cause, an implanted device such as a pacemaker or an implantable cardiac defibrillator may be recommended. In some moderate or severe cases, cardiac resynchronization therapy (CRT) or cardiac contractility modulation may be of benefit. A ventricular assist device (for the left, right, or both ventricles), or occasionally a heart transplant, may be recommended in those with severe disease that persists despite all other measures.

### Prognosis and Survival

Prognosis in heart failure can be assessed in multiple ways, including clinical prediction rules and cardiopulmonary exercise testing. Clinical prediction rules use a composite of clinical factors such as laboratory tests and blood pressure to estimate prognosis. Among several clinical prediction rules for prognosticating acute heart failure, the ‘EFFECT rule’ slightly outperformed other rules in stratifying people and identifying those at low risk of death during hospitalization or within 30 days. Easy methods for identifying people that are low-risk are as follows. ADHERE Tree rule indicates that people with blood urea nitrogen <43 mg/dl and systolic blood pressure at least 115 mm Hg have less than 10% chance of inpatient death or complications. The BWH rule indicates that people with systolic blood pressure over 90 mm Hg, respiratory rate of 30 or fewer breaths per minute, serum sodium over 135 mmol/l, and no new ST-T wave changes have less than 10% chance of inpatient death or complications. An important method for assessing prognosis in people with advanced heart failure is cardiopulmonary exercise testing (CPX testing). CPX testing is usually required prior to heart transplantation as an indicator of prognosis. CPX testing involves measurement of exhaled oxygen and carbon dioxide during exercise. The peak oxygen consumption (V02 max) is used as an indicator of prognosis. In general, V02 max less than 12-14 cc/kg/min indicates poor survival and suggests that the person may be a candidate for a heart transplant. People with a V02 max <10 cc/kg/min have a clearly poorer prognosis. The most recent International Society for Heart and Lung Transplantation guidelines also suggest two other parameters that can be used for evaluation of prognosis in advanced heart failure, the heart failure survival score, and the use of a criterion of VE/VCO2 slope >35 from the CPX test. The heart failure survival score is calculated using a combination of clinical predictors and the V02 max from the CPX test. Heart failure is associated with significantly reduced physical and mental health, resulting in a markedly decreased quality of life. With the exception of heart failure caused by reversible conditions, the condition usually worsens with time. Although some people survive many years, progressive disease is associated with an overall annual mortality rate of 10%. Around 18 of every 1000 persons will experience an ischemic stroke during the first year after diagnosis of HF. As the duration of follow-up increases, the stroke rate rises to nearly 50 strokes per 1000 cases of HF by 5 years. Dr. Malik's research has shown Heart failure is a serious medical disorder associated with high mortality. Mortality rates at 1 year and 5 years are 22% and 43%, respectively. The highest mortality is in patients with advanced NYHA class. In addition, heart failure associated with an MI carries a mortality of 30-40%. Heart failure that is associated with systolic dysfunction has a 50% mortality over 5 years. Further, patients with heart failure need repeated admissions over the years.

In 2011, non-hypertensive heart failure was one of the 10 most expensive conditions seen during inpatient hospitalizations in the U.S., with aggregate inpatient hospital costs more than $10.5 billion. Heart failure is associated with a high health expenditure, mostly because of the cost of hospitalizations; costs have been estimated to amount to 2% of the total budget of the National Health Service in the United Kingdom, and more than $35 billion in the United States. Heart failure hospitalizations are a major financial cost to healthcare systems. This study aimed to evaluate the costs associated with inpatient hospitalization.

### Substance P in Acute and Chronic Heart Conditions

Substance P is a sensory nerve neuropeptide located near coronary vessels in the heart. Substance P may be one of the first mediators released in the heart in response to hypertension and can contribute to adverse myocardial remodeling via interactions with the neurokinin-1 receptor. Substance P promotes cardiac hypertrophy, including the expression of fetal genes known to be re-expressed during pathological hypertrophy. Substance P regulates collagen production and fibrosis. As for methods and results: Spontaneously hypertensive rats (SHR) were treated with the neurokinin-1 receptor antagonist L732138 (5 mg/kg/d) from 8 to 24 weeks of age. Age-matched WKY served as controls. The gene encoding substance P, TAC1, was upregulated as blood pressure increased in SHR. Fetal gene expression by cardiomyocytes was increased in SHR and was prevented by L732138. Cardiac fibrosis also occurred in the SHR and was prevented by L732138. Endothelin-1 was upregulated in the SHR and this was prevented by L732138. In isolated cardiac fibroblasts, substance P transiently up-regulated several genes related to cell-cell adhesion, cell-matrix adhesion, and extracellular matrix regulation, however, no changes in fibroblast function were observed. Basically, substance P activation of the neurokinin-1 receptor induced expression of fetal genes related to pathological hypertrophy in the hypertensive heart. Additionally, activation of the neurokinin-1 receptor was critical to the development of cardiac fibrosis. Since no functional changes were induced in isolated cardiac fibroblasts by substance P, it is concluded that substance P mediates fibrosis via up-regulation of endothelin-1.

Substance P and NKA have long been known to have negative inotropic and chronotropic effects on the normal heart, but it is only recently that it was begun to consider that sensory nerve neuropeptides may have key roles in regulating adverse myocardial remodeling and the subsequent development of heart failure. At least one study has found all his patients had substance P-containing nerve fibers close to arterioles, capillaries, and veins. Substance P-containing nerve fibers have also been found surrounding the adventitia of coronary vessels in atherosclerotic regions of human coronary arteries. In atrial biopsies taken from patients undergoing open-heart surgery. Substance P-containing nerves were identified between cardiomyocytes and around blood vessels. Substance P-containing myocardial nerves include the stellate ganglia, vagus nerve, the T4 region of the spinal cord, the dorsal root ganglia, and the nodose ganglion of the vagus nerve. About 5-10% of coronary artery endothelial cells from rat hearts also contain substance P.

The tachykinin, substance P, is found primarily in sensory nerves. In the heart, substance P-containing nerve fibers are often found surrounding coronary vessels, making them ideally situated to sense changes in the myocardial environment. Recent studies in rodents have identified substance P as having dual roles in the heart, depending on disease etiology and/or timing. Thus far, these studies indicate that substance P may be protective acutely following ischemia-reperfusion but damaging long-term in non-ischemic induced remodeling and heart failure. Sensory nerves may be at the apex of the cascade of events leading to heart failure, therefore, they make a promising potential therapeutic target that warrants increased investigation.

Substance P and NK1-R are involved in the regulation of heart rate, blood pressure, ischemia, reperfusion, cardiac response to stress, and angiogenesis. Substance P is best known as a potent vasodilator. The vasodilatory effects of substance P are dependent on the nitric oxide production of endothelium cells, which leads to smooth muscle relaxation and, ultimately, the dilation of the blood vessel. As a result, intravenous administration of substance P results in decreased blood pressure.

Substance P and NK1 receptors are found in cardiac muscle and may factor into the pathogenesis of myocardial infarction, myocarditis, and reperfusion injury. Interestingly, in a study using a capsaicin-treated heart (a substance that depletes substance P and calcitonin gene-related peptide), acute infarction resulted in more irreversible injury to myocardial tissue than a non-capsaicin treated heart. Therefore, the hypothesis is that substance P and calcitonin gene-related peptide are involved in the reduction of reperfusion injury through the vasodilation of coronary arteries acutely. However, in the long term, substance P also plays a role in cardiac remodeling and fibrosis through the activation of cardiac mast cells and upregulation of endothelium-1 in cardiomyocytes.

Publication by D'Souza and Robinson, which are noted at the end of this section along with other publications, both showed that huge increases in substance P levels in mouse hearts during viral cardiomyopathy infections compared to 71 pg/mg in normal mice in D'Souza's study. Robinson showed a 60-fold increase in substance P during the infection. Robinson went a step further and infected mice that had a gene deletion for the substance P receptor (NK-1). In the knockout mice, substance P levels increased dramatically during the infection like the control mice, but they experienced no cardiac damage from the infections. The control mice suffered moderate to severe cardiac damage and 40% died. The mice without the substance P receptor suffered no deaths or cardiac injury. It seems the virus uses extreme levels of substance P to infect and damage their host and enable them to infect the heart. Without the ability to damage the heart and immune response with an extreme cytokine reaction the virus could not cause an infection or damage.

A study showed that occlusion of the left anterior descending coronary artery resulted in an increase in substance P in the T-4 region of the spinal cord. Spontaneously hypertensive rats had more substance P in the dorsal root ganglia than non-hypertensive rats.

In a study of chronic volume overload induced heart failure, mice with the NK-1 receptor (substance P) deletion gene did not develop heart failure as the controls did.

### SUMMARY

Substance P is involved with two very important mechanisms in the human body. It activates the repair mechanism when a tissue is damaged whatever the cause of the damage maybe. It also increases blood flow, mediates the repair of injured tissues, and stimulates fibrosis to replace damaged or lost tissue. It also activates the immune system to produce cytokines in response to damage or infection. These are vital mechanisms to repair and dispose of injured tissue and initiate cytokine production to mitigate and delay infections until antibody production can begin and eliminate the infection. It works well in most situations.

In acute heart failure there are several situations where substance P can cause pathology. In a situation where too much tissue is destroyed in an injury such as a heart attack, the normal disposal mechanism cannot eliminate the dead tissue quickly enough. Dead and dying cells lyse and toxic intracellular contents are spilled and irritate and damage nerves which causes an acute overproduction of substance P with the resulting extreme cytokine release, which can damage cardiac tissue not only near the site of injury, but across the entire heart. In Myocarditis (viral infections) of the heart, it has been shown that a major part of their pathology of these viruses is their ability to acutely (up to 60× normal levels) trigger substance P overproduction. The resulting extreme cytokine event damage and destroy cardiac tissue.

These two acute manifestations of substance P overproduction can cause cytokine storm-like damage and destruction of cardiac tissue. Chronically elevated levels of Substance P can cause slow, progressive cardiac tissue damage. If after the acute damage of a heart attack or viral infection the substance P levels never returns to normal, then varying levels of chronic cytokine overproduction can occur. This can result in chronic heart failure. How high above normal, and the patient's genetic and physical condition, explains the varying rates of progression of CHF (Chronic Heart Failure).

The following is the inventor's theory of the mechanism of how high blood pressure causes chronic heart failure. High blood pressure causes stretching and damage to the cardiac arteries. This also damages the sensory nerves imbedded in the arteries. This results in chronic substance P production with resulting elevated cytokine and chronic damage to the heart. Valvular problems can also cause CHF. They do this by allowing blood to regurgitate back into the heart with each beat. This increased workload on the heart to maintain adequate blood flow causes chronic inflammation.

The inventor's theory of the cause of the large percentage of patients with heart failure is due to unknown cause. In up to 50% of CHF patients, the cause is unknown. Some patients are in their 20s to 30s. Many of the cases are comorbid with migraines, fibromyalgia, asthma, COPD, diabetes, and cardiac arrhythmia. These conditions are known to be caused by excess glutamate and substance P production from the dorsal root ganglia and vagal ganglia. The inventor theorizes that these combinations are the source of the excess glutamate and substance P production and believe this causes chronically elevated substance P and cytokine damage to the heart.

The source of this overproduction of glutamate and substance P can be an injury directly to the nerves in the heart that innervate the heart, the vagal nerves, or damage to the cardiac arterial nerves due to high blood pressure. Another source is through a process called central sensitization. Central Sensitization is a condition where glutamate and substance P can travel up and down the spinal fluid and irritate other cell bodies in ganglia and cause them to chronically overproduce glutamate (fibromyalgia) and/or substance P immune cytokine activation. As mentioned above, other conditions that are caused by excess levels of glutamate and substance P influence the heart by central sensitization.

### Treatment

### Acute Sudden Heart Injuries

### Cardiac Infarction, Viral Infarction, or Other Physical Injuries to the Heart.

No matter the cause of an acute cardiac injury, be it a viral infection or heart attack, or some other physical injury. The resulting acute levels of substance P with the resulting cytokine release can damage the heart. The inhaled anesthetics such as nitrous oxide treatments should mitigate the acute substance P production and minimize the cytokine induced tissue damage which can be more pathogenic than the causative injury. Nitrous oxide does not cause respiratory depression, over-sedation, or irritation of the lungs at effective dosages. The inhaled dosage can be from 1% nitrous oxide/99% oxygen to 70% nitrous oxide/30% oxygen depending on individual needs and sensitivity. Clinical indications suggest that 40% nitrous oxide/60% oxygen to 50% nitrous oxide/50% oxygen would be optimal. Time of inhalation would vary from one minute to one hour with 20 minutes being the optimal time frame. A longer time may be used if necessary. Duration of substance P suppression can be from 1 minute to 12 hours with average cases of 4-6 hours of substance P suppression. In some embodiments, the nitrous oxide and oxygen may be provided to an adult who weighs about 150 lbs. for about between 1 minute and about 1 hour every about 4-6 hours. Preferably, the nitrous oxide and oxygen may be provided to an adult who weighs about 150 lbs. for about 20 minutes every about 4-6 hours. The nitrous oxide and oxygen may be provided by continuous administration over the period of time.

The nitrous oxide mechanism of action is suppression of substance P production in the peripheral spinal and vagus ganglia. The length of treatment to suppress the severe cytokine reaction will vary and be influenced by the time to medically correct the initiating factor. The nitrous oxide mechanism is suppression of substance P in the peripheral spinal and vagus ganglia. The amount of nitrous oxide, duration of administration, and length of effectiveness will have to be titrated to the individual. For children, adjustments will have to be made for age and body weight. Nitrous oxide or any inhaled anesthetics can be used to mitigate the extreme cytokine reaction in some heart conditions. Other inhaled anesthetics may have to be used at a different oxygen % than the nitrous oxide to produce effective clinical results. Other inhalants can be included in the above-compositions such as for other purposes. This will prevent pathologically elevated levels of cytokines damaging to the heart while the precipitating event is addressed.

Blood test could be used to evaluate substance P levels to titrate nitrous oxide administration and duration. Blood test could also be used to measure inflammatory mediators such as C-reactive protein or sedimentation rates. If side effects from too much substance P suppression nitrous oxide use occur, then inhalation may be reduced or eliminated. Before, during, and after the provision of the inhaled nitrous oxide and oxygen, blood tests may be done to monitor and assess the patient's cytokine level including a substance P level and viral load inflammatory mediators such as C-reactive protein or sedimentation rates. In addition, before, during and after the inhalation of nitrous oxide and oxygen, a blood oxygen level and a pulse may be monitored and assessed. When blood tests show an increase in substance P, glutamate, C-reactive protein, or other inflammatory mediators and/or the symptoms begins to re-develop, more nitrous oxide/oxygen can be given to combat this effect.

If nitrous oxide treatments must be extended over one day, then B-12 and/or folate (folic acid) vitamin supplements (e.g., 300 mcg folic acid sublingual or by injection and/or B-12 1,000 mcg by mouth daily) may need to be administered as long term Nitrous Oxide use can result in depletion of the important vitamins.

### Chronic Heart Failure

In the cases of chronic heart failure, where the source of this chronic production of substance P is the neurostructural cells in the vagus and dorsal root ganglia or from other ganglia by the process of central sensitization. Subcutaneous botulinum toxin by the present disclosure's novel injection techniques and dosages will control this chronic substance P production and the resulting cytokine damage to the heart. The subcutaneous and/or intradermal botulinum toxin would be safe to use with the present disclosure's injection technique, locations, and dosing. Importantly this mechanism only affects the overproduction of substance P and does not affect normal production or mechanisms. This is a huge advantage over the analogs and antagonists that affect substance P. The botulinum toxin only affects the overproduction of glutamate, substance P, and CGRP in the sensory nerve when used in these doses subcutaneously or intradermally, which inhibits the over production of the glutamate, substance P, and CGRP from the sensory nerve, not the intra-nerves glutamate, substance P and CGRP that is excreted out of the terminal of the nerves.

Botulinum toxin can mitigate or control the chronic overproduction of substance P and glutamate in the spinal cranial and vagus nerve ganglia. Preventing the chronic release of substance P and glutamate from these neuro structural cells, can suppress or control the chronic inflammation in the thoracic dermatome (t-1 to t-5) that innervates the heart, which is believed to be a major factor in heart failure, and stop the production of intercellular and intracellular plaque which is the result of improper protein folding due to elevated intercellular pH. It also prevents activation of the embryonic neural pruning mechanism activated by excess intracellular calcium ions.

Botulinum toxins cleave and destroy a protein called synaptosomal nerve-associated protein 25 (“SNAP25”) and/or synaptobrevin (also called vesicle-associated membrane protein (“VAMP”)). Botulinum toxins A, C, and E cleave SNAP25 at different locations, and the destroyed protein cannot function until the cell makes new ones. Botulinum toxins B, D, F, and G cleave VAMP present at the cytoplasmic surface of the synaptic vesicle. The two important locations in the body where the proteins are found are at the terminals of the motor neurons (muscle) and in the cell membranes of astrocytes, glial cells, and satellite cells. These three cell types surround sensory neurons and form part of the blood-brain barrier. In motor nerves, to cause them to fire, vesicles of acetylcholine are moved from inside the motor neuron across the cell membrane at the synapse between the motor nerve and muscle fiber. Acetylcholine is released into the synapse and activated receptors in the muscle fiber, which contracts the muscle fiber. In sensory nerves, when a nerve is damaged from physical or mental injuries, the three aforementioned structural cells produce large amounts of substance P, Calcitonin Gene-Related Peptide (CGRP), and glutamate internally and the molecules are moved by vesicles to the cell membrane where the SNAP25 and/or VAMP moves it through the cell membrane into the cerebral spinal fluid (CSF) that surrounds the neurons. There the molecules bind to the receptor on the sensory nerves, causing the neuro excitatory effects. The molecules can also diffuse in the cerebral spinal fluid and influence other sensory nerves to become hyperactive, a process called central sensitization.

This mechanism of cleaving the SNAP25 and/or VAMP in muscles and sensory nerves causes the only known clinical effects of the botulinum toxins, which paralyzes muscles for 3-4 months until the cell grows a new protein. This effect has been used for decades for overactive muscles (cervical dystonia, blepharospasm, tic, Parkinson's, cerebral palsy, etc.), wrinkles in the face, excessive sweating, and overactive bladder.

In the sensory nerves, botulinum toxin has been used for migraines and depression. The effect of blocking the SNAP25 and/or VAMP in the glial, satellite, and astrocyte cells will remain for 5-9 months until these cells grow their new proteins. The important part of this is the botulinum toxin does not destroy cells and does not stop the normal production or effects of acetylcholine (muscles) or substance P, CGRP, or glutamate in sensory nerves. These facts give huge advantages over a monoclonal antibody which would eliminate all glutamate, CGRP, and substance P. Side effects would be disastrous. The receptor antagonists are also involved and may be problematic. They are not site-specific; they block glutamate, substance P, and CGRP everywhere. Too little glutamate, substance P, and CGRP is as problematic as too much. It is hard to regulate the oral or I.V. doses to obtain the correct reduction in areas that are too high in glutamate, substance P, and CGRP, without over-reduction in areas with normal levels.

The cleaving of the SNAP25 and/or VAMP allows small doses of botulinum toxin to be injected into specific muscles to calm the muscle's overreaction or paralyze the muscles temporarily if that is required. Or, if injected subcutaneously near unmyelinated sensory nerves, it can stop the overproduction of the sensory neuron excitatory compounds without affecting normal glutamate, substance P, and CGRP production and function. It is, however, noted that botulinum toxin is highly lethal. Botulinum toxin is the most toxic poison known. One molecule of botulinum toxin destroys one protein molecule of SNAP25 and/or VAMP. Its production, storage, and injection must be carried out with knowledge and care.

In particular, the mechanism of the sensory effect (stopping overproduction of glutamate, substance P, and CGRP) is as follows: almost all nerves in the human body are surrounded by a protective coating called myelin, which protects the nerve and makes neural conduction faster. It is difficult for botulinum toxin to penetrate the myelin. Just under the skin are some sensory pain nerves called C-fibers, which are unmyelinated. Research has shown that it is much easier for the botulinum toxins to penetrate these axons and diffuse up the axon to the cell body into the CSF and affect the SNAP25 and/or VAMP on the glial, satellite, and astrocyte cells. Subsequently, botulinum toxin destroys the SNAP25 and/or VAMP proteins and prevents the release of the excess substance P, CGRP, and glutamate that is involved in the neural injury response mechanism without affecting normal glutamate, substance P, and CGRP production, use, or receptors. An example of what goes wrong with the normal nerve mechanism is an infection of a nerve by the shingles virus. The infection damages the nerve, but does not kill it, or there would be no feeling (numbness). This causes a spike in the production of glutamate, substance P, and CGRP, which causes the well-known shingles pain and hypersensitivity. Over 2-3 months, the infection is controlled, the nerve heals, and the overproduction of the neuro excitatory chemical gets back to normal. However, sometimes, for unknown reasons, the overproduction does not get back to normal but remains high, with persisting severe chronic pain and hypersensitivity. Chronically overstimulated neurons can cause numerous problems depending on where the neurons are located. The neuron excitatory substances can travel up the spinal cord to the brain in the CSF and affect neurons there. This process is called Central Sensitization. Depending on where glutamate, substance P, and CGRP are produced and where the tachykinins travel to, the tachykinins can cause chronic pain, dyslexia, headaches, vertigo, sensitivity to light, sensitivity to touch, cold sensitivity, overactive bladder, depression, anxiety, flashbacks, mental fogginess, vasoconstriction of extremities, and sleep disturbances.

The subcutaneous or intradermal injection may be administered to and/or around a vicinity of a trigeminal nerve of the patient. The trigeminal nerve is selected from the group consisting of an ophthalmic nerve, maxillary nerve, mandibular nerve, supra orbital nerve, supra trochlear nerve, infraorbital nerve, lacrimal nerve, nasociliary nerve, superior alveolar nerve, buccal nerve, lingual nerve, inferior alveolar nerve, mental nerve, an auriculotemporal nerve, lesser occipital nerve, a greater occipital nerve, and a combination thereof. The subcutaneous or intradermal injection may be administered by subcutaneous or intradermal injection to and/or around a vicinity of a cervical nerve of the patient. The cervical nerve is selected from the group consisting of a c-2 nerve, c-3 nerve, c-4 nerve, c-5 nerve, c-6 nerve, c-7 nerve, c-8 nerve and a combination thereof. The subcutaneous or intradermal injection may be administered to and/or around a vicinity of a nerve selected from the group consisting of a thoracic nerve, a lumbar nerve, and a sacral nerve of the patient. The administering may comprise by subcutaneous or intradermal injection 1-4 units to and/or around the vicinity of an ophthalmic, maxillary, and/or mandibular nerve of the trigeminal nerve (bilateral), 1-4 units to and/or around the vicinity of c-2 to c-3, c-4 to c-6, and/or c-7 to c-8 of the cervical nerve, about one-inch lateral to the spine (bilateral), 1-4 units to and/or around the vicinity of t-2 to t-3, t-5 to t-6, t-7 to t-9, and/or t-10 to t-12 of the thoracic nerve, about one inch lateral to the spine (bilateral), 1-4 unit to and/or around the vicinity of 1-1 to 1-2, 1-2 to 1-3, and/or 1-4 to 1-5 of the lumbar nerve, about one inch lateral to the spine (bilateral), and/or 1-4 units to and/or around the vicinity of s-1 to s-2, s-3 to s-4, and/or s-4 to s-5 of the sacral nerve, about one inch lateral to the spine (bilateral). The administering may comprise by subcutaneous or intradermal injection 2-4 units to and/or around the vicinity of an ophthalmic, maxillary, and/or mandibular nerve of the trigeminal nerve (bilateral), 2-4 units to and/or around the vicinity of c-2 to c-3, c-4 to c-6, and/or c-7 to c-8 of the cervical nerve, about one-inch lateral to the spine (bilateral), 2-4 units to and/or around the vicinity of t-2 to t-3, t-5 to t-6, t-7 to t-9, and/or t-10 to t-12 of the thoracic nerve, about one inch lateral to the spine (bilateral), 2-4 unit to and/or around the vicinity of 1-1 to 1-2, 1-2 to 1-3, and/or 1-4 to 1-5 of the lumbar nerve, about one inch lateral to the spine (bilateral), and/or 2-4 units to and/or around the vicinity of s-1 to s-2, s-3 to s-4, and/or s-4 to s-5 of the sacral nerve, about one inch lateral to the spine (bilateral).

Blood test could be used to evaluate substance P or inflammatory mediator levels to titrate botulinum toxin administration and duration. Before, during, and after the provision of the botulinum toxin, blood tests may be done to monitor and assess the patient's cytokine level including a substance P level. In addition, before, during and after the administration of botulinum toxin, a blood oxygen level and a pulse may be monitored and assessed. When blood tests show an increase in substance P and/or the symptoms begins to re-develop, more botulinum toxin can be given to combat this effect. If levels/symptoms fail to normalize, nitrogen oxide may be administered to help lower substance P levels without producing side effects.

The botulinum toxin is selected from the group consisting of botulinum toxin type A, botulinum toxin type B, botulinum toxin type C, botulinum toxin type D, botulinum toxin type E, botulinum toxin type F, and botulinum toxin type G, a fragment thereof, a hybrid thereof, a chimera thereof, and a combination thereof. In general, the therapeutically effective amount of the botulinum toxin administered is between about 1 unit and about 150 units. The therapeutically effective amount can be about 1 to about 50 units, about 1 to about 30 units, about 50 to about 100 units, about 1 to about 60, about 6 to about 60, and about 50 to about 150. With the novel administration methods described herein, the use of as little as 60 units should be effective in treating chronic heart failure. In some embodiments, a total dosage of the botulinum toxin to an adult who weighs about 150 lbs. is less than or equal to about 50 units, and the total dosage of the botulinum toxin in an adult is adjusted for weight. The dosage can be adjusted to the subject's body weight. If levels/symptoms fail to normalize, then if desired, a small dose of one of the glutamate antagonists can be administered to help lower glutamate levels without producing side effects.

Botulinum toxins for use according to the present disclosure can be stored in lyophilized, vacuum dried form in containers under vacuum pressure or as stable liquids. Prior to lyophilization, the botulinum toxin can be combined with pharmaceutically acceptable excipients, stabilizers and/or carriers, such as albumin. The lyophilized material can be reconstituted with saline or water to create a solution or composition containing the botulinum toxin to be administered to the patient.

Preferably, the botulinum neurotoxin is peripherally administered by injecting it into or in the vicinity of the aforementioned nerve or to the aforementioned nerve branch or its ganglion nuclei. This method of administration permits the botulinum neurotoxin to be administered to and/or to affect select intracranial target tissues. Methods of administration include injection of a solution or composition containing the botulinum neurotoxin, as described above, and include implantation of a controlled release system that controllably releases the botulinum neurotoxin to the target trigeminal tissue. Such controlled release systems reduce the need for repeat injections. Diffusion of biological activity of a botulinum toxin within a tissue appears to be a function of dose and can be graduated. Thus, diffusion of botulinum toxin can be controlled to reduce potentially undesirable side effects that may affect the patient's cognitive abilities. For example, the botulinum neurotoxin may be administered so that it primarily affects neural systems believed to be involved in a selected neuropsychiatric disorder and does not have negatively adverse effects on other neural systems.

In addition, the botulinum neurotoxin may be administered to the patient in conjunction with a solution or composition that locally decreases the pH of the target tissue environment. For example, a solution containing hydrochloric acid may be used to locally and temporarily reduce the pH of the target tissue environment to facilitate translocation of the neurotoxin across cell membranes. The reduction in local pH may be desirable when the composition contains fragments of botulinum neurotoxins that may not have a functional targeting moiety (e.g., a portion of the toxin that binds to a neurotoxin receptor), and/or a translocation domain). By way of example, and not by way of limitation, a fragment of a botulinum toxin that comprises the proteolytic domain of the toxin may be administered to the patient in conjunction with an agent that decreases the local pH of the target tissue. Without wishing to be bound by any particular theory, it is believed that the lower pH may facilitate the translocation of the proteolytic domain across the cell membrane so that the neurotoxin fragment can exert its toxic effects within the cell. The pH of the target tissue is only temporarily lowered so that neuronal and/or glial injury is reduced.

Long term nitrous oxide treatment may have toxic side effects, and folic acid and/or B-12 should be administered. The folic acid and/or B-12 can be administered prior to beginning the treatment period (multiple treatments over time) and/or during or after the treatment period. The physician can require that the patient take the folic acid and/or B-12 and in some case, can conduct tests to determine compliance. Compliance can be performed in other ways such as by the physician asking questions or other step (or combination of steps).

The embodiments of the invention are further described in the following prophetic examples. These examples are for illustrative purposes only, and are not to be construed as limiting the appended claims.

### Example 1

A 77 year old female patient suffers from acute heart failure caused by viral infection. The patient weighs about 157 lbs. The patient primarily has an issue with shortness of breath and peripheral edema. She first receives antiviral agents and then receives 50% nitrous oxide/50% oxygen by inhalation 20 minutes every 4 hours for a week. After the botulinum toxin administration, the patient reports significant improvement in respiratory rate and peripheral edema, and substance P and glutamate blood levels are measured to be lower than before. The patient optionally receives additional treatments such exercises and dietary changes. The patient also receives B-12 and folate vitamin supplements.

### Example 2

A 82 year old male patient suffers from chronic heart failure caused by systolic dysfunction. The patient weighs about 163 lbs. The patient primarily has an issue with shortness of breath and fatigue. He receives botulinum toxin in the area of trigeminal, cervical, thoracic, lumbar and sacral nerves (2 units in ophthalmic, 2 units in maxillary, 2 units in mandibular of trigeminal nerve bilaterally; 2 units in the c-2-c-3, 2 units in the c-5-c-6, 2 units in the c-7-c-8 of cervical nerve bilaterally; 2 units in the t-1-t-3, 2 units in the t-5-t-6, 2 units in the t-8-t-9, 2 units in the t-11-t-12 of thoracic nerve bilaterally; 2 units in the 1-1-1-2, 2 units in the 1-3-1-4, 2 units in the 1-4-1-5 of lumbar nerve bilaterally; 2 units in the s-1-s-2, 2 units in the s-3-s-4, 2 units in the s-5 of sacral nerve bilaterally for a total of not more than 60 units). After the botulinum toxin administration, the patient reports significant improvement in breath and fatigue. Substance P blood levels are measured to be lower than before. The patient optionally receives additional treatments such exercises and dietary changes.

### Example 3

A 78 year old female patient suffers from chronic heart failure caused by diastolic dysfunction. The patient weighs about 152 lbs. The patient primarily has an issue with shortness of breath, fatigue, and pulmonary edema. She receives botulinum toxin in the area of trigeminal, cervical, thoracic, lumbar and sacral nerves (1 units in ophthalmic, 1 units in maxillary, 1 units in mandibular of trigeminal nerve bilaterally; 1 units in the c-2-c-3, 1 units in the c-5-c-6, 1 units in the c-7-c-8 of cervical nerve bilaterally; 1 units in the t-1-t-3, 1 units in the t-5-t-6, 1 units in the t-8-t-9, 1 units in the t-11-t-12 of thoracic nerve bilaterally; 1 units in the 1-1-1-2, 1 units in the 1-3-1-4, 1 units in the 1-4-1-5 of lumbar nerve bilaterally for a total of not more than 30 units). After the botulinum toxin administration, the patient reports significant improvement in breath and pulmonary edema Substance P and glutamate blood levels are measured to be lower than before. The patient optionally receives additional treatments such exercises and dietary changes.

The description of ranges also describes the ranges within the specifically described range and describes individual numerical points for treatment. The described ranges are inclusive of endpoints in the range.

It should be understood that the present description of embodiments of the invention includes a composition for use in treating the conditions.

The usage of terms “may,” “can,” or similar terms herein are only for the purpose of flexibility in the disclosure and include, but are not limited to, the meaning of “is,” “are,” or similar terms.

It should be understood that the above description of embodiments of the invention and specific examples, while indicating preferred embodiments of the present invention, are given by way of illustration and not limitation. Many changes and modifications within the scope of the present invention may be made without departing from the spirit thereof, and the present invention includes all such changes and modifications.

What is claimed is:

1. A method of treating chronic heart failure in a patient in need thereof, comprising:
administering a botulinum toxin to the patient by subcutaneous or intradermal injection, 1-4 units to and/or around the vicinity of a trigeminal nerve, 1-4 units to and/or around the vicinity of a cervical nerve, lateral to the patient's spine, 1-4 units to and/or around the vicinity of a thoracic nerve, lateral to the spine, 1-4 units to and/or around the vicinity of a lumbar nerve, lateral to the spine, and/or 1-4 units to and/or around the vicinity of a sacral nerve, lateral to the spine, thereby treating chronic heart failure.

2. The method of claim 1, wherein the trigeminal nerve is selected from the group consisting of an ophthalmic nerve, maxillary nerve, mandibular nerve, supraorbital nerve, supratrochlear nerve, infraorbital nerve, lacrimal nerve, nasociliary nerve, superior alveolar nerve, buccal nerve, lingual nerve, inferior alveolar nerve, mental nerve, an auriculotemporal nerve, a lesser occipital nerve, a greater occipital nerve, or a combination thereof.

3. The method of claim 1, wherein the cervical nerve is selected from the group consisting of a c-2 nerve, c-3 nerve, c-4 nerve, c-5 nerve, c-6 nerve, c-7 nerve, c-8 nerve, or a combination thereof.

4. The method of claim 1, wherein the thoracic nerve is selected from the group consisting of a t-2 nerve, t-3 nerve, t-5 nerve, t-6 nerve, t-7 nerve, t-8 nerve, t-9 nerve, t-10 nerve, t-11 nerve, t-12 nerve and a combination thereof.

5. The method of claim 1, wherein the lumbar nerve is selected from the group consisting of a 1-1 nerve, 1-2 nerve, 1-3 nerve, 1-4 nerve, 1-5 nerve and a combination thereof.

6. The method of claim 1, wherein the sacral nerve is selected from the group consisting of a s-1 nerve, s-2 nerve, s-3 nerve, s-4 nerve, s-5 nerve and a combination thereof.

7. The method of claim 1, wherein the botulinum toxin is selected from the group consisting of botulinum toxin type A, botulinum toxin type B, botulinum toxin type C, botulinum toxin type D, botulinum toxin type E, botulinum toxin type F, and botulinum toxin type G, a fragment thereof, a hybrid thereof, a chimera thereof, or a combination thereof.

8. The method of claim 1, wherein the therapeutically effective amount of the botulinum toxin administered is between about 1 unit and about 60 units.

9. The method of claim 1, wherein a total dosage of the botulinum toxin to an adult who weighs about 150 lbs. is less than or equal to about 50 units, and the total dosage of the botulinum toxin in an adult is adjusted for weight.

10. The method of claim 1, wherein each of the subcutaneous or intradermal injections is bilateral.

11. A method of treating acute heart failure in a patient in need thereof, the method comprising:
administering nitrous oxide and oxygen to the patient by inhalation, and
administering B-12 and/or folic acid to the patient, thereby treating acute heart failure.

12. The method of claim 11, wherein a composition of the nitrous oxide and oxygen comprises from about 1% nitrous oxide/about 99% oxygen to about 70% nitrous oxide/about 30% oxygen.

13. The method of claim 12, wherein the composition of the nitrous oxide and oxygen comprises from about 40% nitrous oxide/about 60% oxygen to about 50% nitrous oxide/about 50% oxygen.

14. The method of claim 13, wherein the composition of the nitrous oxide and oxygen comprises about 50% nitrous oxide/about 50% oxygen.

15. The method of claim 11, wherein the nitrous oxide and oxygen are provided to an adult who weighs about 150 lbs. for about between 1 minute and about 1 hour every about 4-6 hours.

16. The method of claim 15, wherein the nitrous oxide and oxygen are provided to an adult who weighs about 150 lbs. for about 20 minutes every about 4-6 hours.

17. The substance of claim 15, wherein a composition, duration, an interval, and a total amount of the nitrous oxide and oxygen provided to an adult, or a child is adjusted for age, weight, or a combination thereof.

18. The method of claim 13, wherein the nitrous oxide and oxygen are provided to an adult who weighs about 150 lbs. for about between 1 minute and about 1 hour every about 4-6 hours.

19. The method of claim 18, wherein the nitrous oxide and oxygen are provided to an adult who weighs about 150 lbs. for about 20 minutes every about 4-6 hours.

20. The method of claim 11, wherein the nitrous oxide and oxygen are provided by continuous administration over the period of time.
